(19) United States
(12) Patent Application Publication
(10) Pub. No.: US 2024/0288596 A1
(43) Pub. Date: Aug. 29, 2024

(54) MEDICAL ENGINEERING APPARATUS WITH DETERMINING UNIT FOR DETERMINING A DISTANCE

(71) Applicant: Siemens Healthineers AG, Forchheim (DE)

(72) Inventor: Ulrich Batzer, Spardorf (DE)

(21) Appl. No.: 18/589,689

(22) Filed: Feb. 28, 2024

(30) Foreign Application Priority Data
Feb. 28, 2023 (DE) 10 2023 201 814.4

(51) Int. Cl. G01T 1/29; G01R 15/12; G01T 1/24

(52) U.S. Cl. CPC G01T 1/2978; G01R 15/12; G01T 1/241; G01T 1/244

(57) ABSTRACT

A medical engineering apparatus includes a functional unit that may move relative to an object. The medical engineering apparatus includes a first electrode unit and a second electrode unit arranged separately from the first electrode unit that together form a capacitive sensor unit for determining a distance between the object and the second electrode unit. The first electrode unit is electrically connectable to the object, and the second electrode unit is connected to the functional unit. An electrical alternating signal may be applied to the first electrode unit or the second electrode unit. The medical engineering apparatus includes a determining unit for determining the distance based on a current measured at the first electrode unit or the second electrode unit. The medical engineering apparatus includes an output unit for outputting an output value based on the measured current.

### BACKGROUND

This application claims the benefit of German Patent Application No. DE 10 2023 201 814.4, filed on Feb. 28, 2023, which is hereby incorporated by reference in its entirety.

The present embodiments relate to determining a distance between an object and a functional unit.

Medical engineering apparatuses are known in a wide variety of embodiments (e.g., in imaging or therapy). For example, C-arc or C-arm systems are used for imaging in interventional procedures. Here, the C-arm, for example, moves in the environment of the examination object. A collision or a very short distance between the C-arm and the examination object are to be avoided.

C-arms of X-ray systems and other parts of large medical devices may move in the proximity of the patient or examination object. C-arms and the other parts may undesirably collide with the patient in the process. This may result in injuries to the patient.

Nowadays, responsibility for a collision-free journey (e.g., of the C-arc) often still lies with the operator or user. Until now, the systems or the medical engineering apparatuses have, as a rule, included only measures for collision detection that, however, may often only detect severe collisions (e.g., only stop after the collision).

Some systems include measures for collision avoidance, and this provides stopping before a potential collision. None of these systems for collision avoidance have a single-fault protected design, and are thus not risk-reduction measures, but purely a convenience function that may not be depended upon.

Publication U.S. Pat. No. 8,269,176 discloses a gamma camera with at least one radiation detector head. At least one such radiation detector head includes a large number of capacitive elements that are arranged over at least one radiation-sensitive portion of the radiation detector head. A proximity sensor monitor is coupled to the large number of capacitive elements to detect the proximity of an object to the radiation detector head based on a measured electric property of the capacitive elements. A collision sensor monitor is coupled to the large number of capacitive elements to detect a conductive electric current that flows between spaced-apart, parallel conductive plates of the capacitive element and reacts to a mechanical deformation of the distance between the plates.

It is precisely with regard to more extensive automation of clinical processes that the known measures are not adequate. A collision with the patient or the examination object is to be avoided on automatic repositioning of the C-arm.

Previous measures may often only distinguish between the patient or the examination object and other conductive articles with difficulty. Different behavior (e.g., different safe distances) is desired between examination object and articles. It may be advantageous if the sensor does not detect pillows and the like, or disregards pillows and the like, and if conductive articles, such as the patient couch, as well as the patient or the examination object itself are detected.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a medical engineering apparatus, a method, and a computer program product that enable determination of a distance between an object and a functional unit, and therewith provide collision avoidance, are provided.

Independent of the grammatical term usage, individuals with male, female, or other gender identities are included within the term.

The present embodiments relate to a medical engineering apparatus including a functional unit that may move relative to an object (e.g., an examination object). The medical engineering apparatus includes a first electrode unit and a second electrode unit arranged separately therefrom, which together form a capacitive sensor unit for determining a distance between the object and the second electrode unit. The first electrode unit is configured to be electrically connected to the object, and the second electrode unit is configured in connection with the functional unit. An electrical alternating signal may be applied to the first electrode unit or the second electrode unit. The medical engineering apparatus includes a determining unit for determining the distance based on a current measured at the first electrode unit or the second electrode unit. The determining unit is connected to the first electrode unit and/or the second electrode unit. The medical engineering apparatus includes an output unit for outputting an output value based on the measured current.

The medical engineering apparatus has a functional unit that may move relative to an object (e.g., examination object). The object may be, for example, the examination object (e.g., the patient). The object may be an article in the proximity or be part of the medical engineering apparatus. Determining a distance from the surrounding objects may be regarded as a starting point to prevent collisions actively and independently by the system.

The medical engineering apparatus has a first electrode unit and a second electrode unit arranged distinctly or separately therefrom. The first electrode unit and the second electrode unit may be arranged so as to be spaced apart (e.g., be configured separately from one another at a distance). The first electrode unit and the second electrode unit may be configured so the first electrode unit and the second electrode unit move relative to one another, so the distance may change, for example, on a positioning of the object. The first electrode unit or the second electrode unit may be configured as a receiver unit that is configured to measure a current (e.g., an amplitude of a current). The first electrode unit and/or the second electrode unit may be planar. The first electrode unit and the second electrode unit are arranged separately from one another. The first electrode unit and the second electrode unit together form a capacitive sensor unit for determining a distance between the object or the first electrode unit and the second electrode unit. The conductive connection of the first electrode unit to the object provides the object may act as a first electrode unit, or the distance between the surface of the object and the second electrode unit or the associated surface of the functional unit or of a different object may be determined.

A current, which may be based, for example, on a common mode interference, may be measured by the receiver unit (e.g., the first electrode unit or the second electrode unit). The current may also be referred to as an interference signal. In an embodiment, an ECG recording system may be configured as a receiving unit. The common mode interferences may be caused by a voltage source (e.g., the transmitter unit) in the proximity of the object or the examination object. The object may provide a path to ground. A current (e.g., its amplitude) may be measured. In short, a voltage may be applied at the transmitter unit, which may be registered at the receiver unit by a current measurement, if the transmitter unit and the receiver unit move closer. The closer the transmitter and receiver unit are, the greater the amplitude of the current may be. A distance may be determined or estimated from the amplitude by the determining unit.

The first electrode unit is configured so the first electrode may be electrically connected or coupled to the object. The second electrode unit is configured in connection with the functional unit. An electrical alternating signal may be applied to the first electrode unit or the second electrode unit (e.g., the transmitter unit).

The medical engineering apparatus has a determining unit for determining the distance based on a current measured at the first electrode unit or the second electrode unit. The determining unit is connected to the first electrode unit or the second electrode unit. The determining unit may be connected, for example, to the receiver unit. The connection may be wired or wireless (e.g., via a radio link).

The determining unit may determine a current using a current measuring unit, may determine a distance between a transmitter unit and a receiver unit using a distance determining unit, and/or determine or identify a transmitter unit using an identification unit.

The medical engineering apparatus includes an output unit for outputting an output value based on the measured current (e.g., the distance). The output value may be formed as a warning, a value with respect to the distance, or a control signal (e.g., for stopping a movement of the functional unit). A collision of the functional unit with the object may be prevented based on the output value. For example, the functional unit may be stopped when a threshold value is undershot (e.g., with respect to the distance). In one embodiment, the threshold value may be formed such that a safety distance of several centimeters (e.g., 5 to 10 cm) is preserved even after the functional unit has been stopped. The warning may be output, for example, acoustically or optically. The warning may be output, for example, by a warning sound or a warning announcement. The warning may be output, for example, in a color-coded manner. For example, a region on a user interface may be colored green as long as the functional unit is at a safe distance. As soon as the functional unit moves closer to the object, the region may be colored yellow. As soon as stoppage of the functional unit is necessary or has already been initiated, the region may be colored red. The functional unit may be automatically stopped by a control signal based on the measured current when a distance is undershot.

As a rule, known systems include sensors that are located fully on the moving object. The solution consists in a capacitive collision avoidance system being mounted between the functional unit (e.g., the C-arm) and the object (e.g., the examination object or the patient) instead of solely on the functional unit or the C-arm.

Conventional capacitive sensor systems function such that it is either ascertained how much current a capacitive transmitter is supplying, depending on how much this current may discharge to ground, or how strong the overcoupling of a capacitive transmitter is on a receiver, which is integrated with the transmitter in a unit. In one embodiment, the amplitude of the current may be measured by a current measuring unit. In addition to the amplitude, it is also possible to measure a frequency shift or phase shift and use the frequency shift or the phase shift for determining the distance.

In an embodiment, the proposed construction of the medical engineering apparatus may provide the relevant parts of the functional unit or the C-arm with one or more conductive surface(s) to which one or more transmitter(s) may apply a signal with a frequency of less than 1 MHz. It is possible for the receiver to not be integrated in the functional unit or the C-arm, however. Instead, the object or the patient (e.g., entire patient) may become the receiver. Via an appropriate sensor system on the patient or the object, it is possible to detect how strong the overcoupling of the emitted signals is on the patient. This is stronger if the functional unit or the C-arm with the transmitter unit moves closer to the patient or the object as the receiver unit.

In an embodiment, the transmitter or the transmitter units may be placed around the functional unit or the C-arm. The object or the patient may be made into the receiving antenna or receiver unit in different ways.

A receiving pad may be placed under the object or the patient. This does not have to have particularly good contact with the object or the patient and may also have a distance of a few cm from the object or the patient. The receiving pad is to be completely covered by the object, however, and be shielded in all other directions. In this way, the receiving pad is capacitively coupled to the object or the patient and receives signals (e.g., all signals) that may act on the object or the patient. The receiving pad may be integrated in the patient couch. The patient or the object may be connected to an ECG recording system (e.g., as the receiver unit).

Current that flows through the coupling into the receiver unit to a shared potential of transmitter unit and receiver unit may be measured in or on the receiver unit. The potential (e.g., shared potential) may be the ground potential. The potential may alternatively be a different shared potential that is conducted in the device through corresponding wires.

In a further embodiment, the transmitter units may be even more finely distributed (e.g., around (further) objects that are located in the measuring region).

In one embodiment, an inexpensive solution to collision avoidance between patient and device may be enabled. In one embodiment, it is possible to distinguish between a possible collision with the patient or the examination object or the surroundings or other objects in the surroundings (e.g., by using different impressed frequencies or codes on the signal). In one embodiment, a separate path and therewith, in combination with a conventional collision avoidance system, a possible second path that may enable single-fault safety may be provided. In one embodiment, the collision detection may be very precise and may respond earlier than, for example, force-based methods.

According to one aspect of the present embodiments, the first electrode unit is configured as a transmitter unit, and the second electrode unit is configured as a receiver unit. The first electrode unit, which may be configured so the first electrode unit may be electrically connected to the object or is connected to the object, may be configured as a transmitter unit. The second electrode unit, which is configured in connection with the functional unit, may be configured as a receiver unit. The patient or the examination object or the object may be configured, for example, via a pad to form the transmitter and the functional unit to form a receiver that is connected to the receiver unit.

The receiver unit may include a current measuring unit. The current flowing from an internal reference potential via a capacitive coupling to an external fixed reference potential (e.g., the ground potential) may be measured with the current measuring unit. The receiver unit may have an extensive conductive surface for this purpose. The receiver unit may form a kind of capacitor surface to the fixed reference potential (e.g., the ground potential) or be fastened to a capacitor. A defined signal path may be made available for the capacitively coupled signal thereby. The current measuring unit may be wired in the signal path between the internal reference potential and the ground potential.

In one embodiment, an electrical alternating signal may be applied to the transmitter unit. The electrical alternating signal may also be referred to as a signal.

In one embodiment, the transmitter and receiver unit may be compactly arranged on the functional unit and on the object and still enable a high level of accuracy when measuring the distance measurement.

According to one aspect of the present embodiments, the first electrode unit is configured as a receiver unit and the second electrode unit is configured as a transmitter unit. The first electrode unit, which may be configured so the first electrode unit may be electrically connected to the object or is connected to the object, may be configured as a receiver unit. The second electrode unit, which is configured in connection with the functional unit, may be configured as a transmitter unit.

According to one aspect of the present embodiments, the receiver unit may be changed into a transmitter unit, and the transmitter unit may be changed into a receiver unit. In one possible embodiment, each transmitter may be changed to form a receiver, and each receiver may be changed to form a transmitter. In an embodiment, a transmitter unit may be changed into a receiver unit, and a receiver unit may be changed into a transmitter unit. In one embodiment, the medical engineering apparatus may be adapted to different requirements. For example, when a further object is incorporated (e.g., from third-party manufacturers), an additional receiver unit may be provided by a pad attached to the object, so the distance of the further object from the examination object or functional unit may also be ascertained.

According to one aspect of the present embodiments, the functional unit is an imaging unit (e.g., a C-arm with an X-ray source and an X-ray detector) or a radiation unit. The medical engineering apparatus may be configured, for example, as a medical imaging system. In one embodiment, the medical engineering apparatus may be configured as a medical X-ray imaging system. The medical engineering apparatus may, for example, be configured as an angiography system.

The problem is particularly relevant with regard to the avoidance of collisions in the case of medical engineering apparatuses that have a recording arrangement, with an X-ray source and an X-ray detector, that may be freely positioned in the space. Different directions of projection or, generally, recording geometries that are matched to the medical procedure to be carried out may be implemented here. Medical engineering apparatuses of this kind frequently have a C-arc or C-arm on which the X-ray source and the X-ray detector are arranged facing each other. The C-arc forms a carrier component. The C-arm may, for example, be displaceably mounted by a mount (e.g., a carriage) that forms further carrier components in order to implement an orbital rotation as a degree of freedom of movement in an external arc. The external arc may partially also be referred to as a telescopic C-arc (e.g., an outer telescopic C-arc), and the actual C-arc may be referred to as an inner C-arc. Other embodiments of such medical engineering apparatuses with a C-arc and fixings, which move the X-ray source and the X-ray detector separately, may also include robotic arms. Medical engineering apparatuses of this kind are frequently also referred to as angiography systems (e.g., robotic angiography systems) and are frequently also used in the case of, for example, minimally invasive procedures on the patient or examination object (e.g., generally during clinical interventions). In one embodiment, a collision of the C-arm or C-arc with the object may be avoided, and the C-arm may be automatically positioned.

The radiation unit may include a radiation source for treating the examination object (e.g., in the case of cancers). In a further embodiment, the functional unit may include a robotic unit for removing tissue or for carrying out an interventional procedure.

According to one aspect of the present embodiments, the determining unit determines an overcoupling strength or/and a current discharging to ground. The determining unit may include, for example, a current measuring unit. The determining unit or the current measuring unit may determine, for example, an amplitude of the current. The determining unit may also determine a phase shift or a frequency shift.

According to one aspect of the present embodiments, a plurality of second electrode units are configured in connection with the functional unit. Each second electrode unit of the plurality of second electrode units is configured as a conductive element. The conductive element may be a conductive wire, a conductive track, or a conductive surface. The conductive elements may also be referred to as separate transmitter units or separate receiver units. In general, both the first and the second electrode units may be configured as a conductive element (e.g., an electrically conductive element). For example, the second electrode units on the functional unit may be configured as a plurality of conductive surfaces, tracks, or wires. The second electrode units may be configured along the surface of the functional unit. The second electrode unit may be permanently connected to the functional unit. In an alternative embodiment, the second electrode units may be detachably connected to the functional unit (e.g., in order to retrofit a functional unit or another object with the second electrode unit). The second electrode units may be configured separately or distinctly (e.g., the second electrode units do not form a continuous shared conductive surface). The second electrode units may be configured, for example, on a region of the surface of the functional unit, which may face the object during a course of movement. Regions of the surface of the functional unit, which cannot face the object during a possible course of movement (e.g., for mechanical reasons) may be configured on the surface without second electrode units. Alternatively, regions of the surface of the functional unit, which cannot face the object during a possible course of movement (e.g., for mechanical reasons) may be fitted with second electrode units to avoid, for example, collisions with further objects. In one embodiment, collisions of the medical engineering apparatus with an object or a plurality of objects may be avoided.

According to one aspect of the present embodiments, a signal (e.g., a voltage) is applied to the first electrode unit or the at least one second electrode unit. The first electrode unit or the at least one second electrode unit may be configured, for example, as a conductive element. The signal may be, for example, a voltage or an electrical alternating signal. In one embodiment, the first electrode unit may be configured, for example, as a transmitter unit to which a signal (e.g., a voltage) may be applied. In another embodiment, a signal (e.g., a voltage) may be applied to the at least one second electrode unit, and the unit may act as a transmitter unit or transmitter units. In one embodiment, the signal may be used as a starting point for determining the distance.

The coupling of individual transmitter units or their signals may be distinguished from one another in the receiver unit in that different frequencies or impressed codes are used. In this way, it may be qualitatively determined which transmitter unit is closer to the patient, and an estimation of the orientation of the device may be performed as a result. If the transmitter units are distributed, for example, along the surface of the C-arm, then based on the coupled signal in the receiver unit, it is possible to determine which of the transmitter units of the receiver unit is closest to the examination object. From this, it is possible to infer which region of the surface of the C-arm is closest to the examination object. A position or orientation of the C-arm may be estimated based on this determined region. In one embodiment, the orientation of the C-arm may be inferred from the distinguishability of the transmitter unit. By way of example, a plurality of transmitter units may be arranged along the surface of the C-arm. It may be possible for a signal with a different frequency or a different code to be applied to each transmitter unit of the plurality of transmitter units. A superimposed signal may be received by the receiver unit, and the individual coupled signal components may be distinguished and evaluated.

According to one aspect of the present embodiments, signals of different frequency or with different impressed codes are applied to electrode units configured as separate transmitter units. To be able to distinguish the transmitter units particularly easily, different frequencies (e.g., individual frequencies) or impressed codes may be used for different transmitter units. Binary codes (e.g., sequences of 0 and 1), an M-sequence, or a CDMA method may be used as codes. In one embodiment, different transmitter units may be distinguished from one another based on the measured current or signal.

Each second electrode unit or each transmitter unit may be connected to an impressing unit. Signals of different frequency or with different impressed codes are applied or may be applied to different transmitter units.

The M-sequence is a special form of a binary code. The M-sequence is also referred to as a Maximum Length Sequence (MLS). The M-sequence is a pseudorandom, binary sequence of numbers. A sequence of maximum length is a polynomial ring. The M-sequence may have the following properties: the number of binary ones can be greater by exactly one than the number of binary zeroes. Alternatively, it is possible for pseudorandom sequences generated by computers to not be subject to this restriction. In addition, the lengths of the sections with equal values (e.g., successive zeroes or successive ones) may have a certain ratio to each other: half of length 1, a quarter of length 2, an eighth of length 3, etc. In addition, the autocorrelation and the cross-correlation of the sequences may be periodic and binary.

The CDMA method may also be referred to as the code division multiple access method. The CDMA method is a multiple access method that enables the simultaneous transmission of different usable data streams on a shared frequency range. For distinction, the specific spreading codes are coded as a signal. It is possible for this sequence of codes to also have particular properties. These properties may include, for example, orthogonality or a pseudorandom basis. In one embodiment, superimposed, received signals or currents may be separated and distinguished from one another in the receiver unit.

According to one aspect of the present embodiments, the signal has a frequency less than 1 MHz (e.g., less than 100 Hz). The frequency may also lie in the order of magnitude of 1 Hz. A frequency of substantially 50 Hz or/and 60 Hz may be ruled out. In one embodiment, the frequency differs from other frequencies occurring in the surroundings, so a frequency determined at the receiver may be unequivocally associated with a transmitter unit. For example, low frequencies may be used, whereby a purely electrical field is formed.

According to an aspect of the present embodiments, the object is an examination object. According to one aspect of the present embodiments, the first electrode unit is arranged as a pad under the object or on the object, integrated as a pad in a patient couch, or is configured as a flexible pad. Using a flexible pad, a third party system or a different object or an examination object may be fitted and be connected with a first electrode unit, and thus, the object may come to be formed as a transmitter or receiver unit. The pad may be configured, for example, as an at least partially electrically conductive element that, depending on the arrangement in the proximity of the object, may be rigid or flexible.

Further, it is possible to also embody other device parts as receiving paths (e.g., using a receiving unit) and thus also avoid the collision of device parts among themselves. Flexible receiving pads or flexible pads, which may also be placed on devices from third-party manufacturers, enable universal applicability and expandability of the collision protection

According to one aspect of the present embodiments, the first electrode unit is incorporated by an ECG recording system. The determining unit may be incorporated by the ECG recording system. In one embodiment, an ECG recording system may also provide the function of the first electrode unit. The ECG recording system may be configured as a transmitter or receiver unit. The interference signal path does not have to have a signal measuring cable here. Instead, the interference signal path may correspond to a capacitive measurement or coupling to ground.

For the case of measuring the current with the ECG recording system, the system may be configured with an expanded circuit, similar to in DE 10 2019 203 627 A1, for this purpose. The ECG recording system includes an interference signal path with a current measuring unit. This current measuring unit measures the current flowing from an internal reference potential of the ECG device via a capacitive coupling to an external, fixed reference potential (e.g., the ground potential). The capacitive coupling between the ECG recording system and the ground potential may always be present anyway. To make a defined signal path available for this interference signal, at which the interference signal may be easily measured, a more extensive conductive surface (e.g., in the form of a metal plate or a foil) may be connected to the internal reference potential of the ECG recording system, which forms a “capacitor surface” to the ground potential. Alternatively, this signal path may be implemented by a capacitor component. The current measuring unit may be wired in this interference signal path between the internal reference potential and the ground potential.

For the current measuring unit, a current sensing resistor (e.g., a shunt resistor) wired between internal reference potential and the ground potential, and a voltage measuring facility wired parallel to the shunt resistor, may be used for measuring the current on the interference signal path. The voltage measuring facility may again be implemented by an amplifier (e.g., by a PGA).

The measured interference signal or the measured current may be detected by an interference signal detecting unit wired to the output of the voltage measuring facility (e.g., may be digitized by an A/D converter and optionally be processed further).

According to one aspect of the present embodiments, the medical engineering apparatus also has a conductive layer on the functional unit, to which a second current may be applied, for collision detection. In a further embodiment, individual parts or, for example, all parts of the medical engineering apparatus that may come into contact with the object or the patient are fitted with a conductive layer to which a current in the nA range is applied. As soon as this conductive layer touches the object or the patient, a receiving path may register this immediately and thus detect a collision very quickly. In one embodiment, collisions may be detected very quickly.

The present embodiments also relate to a first electrode unit and to a separately embodied functional unit having a second electrode unit. The two units may together form the medical engineering apparatus. The functional unit may move relative to an object (e.g., an examination object). The second electrode unit is configured in connection with the functional unit. An electrical alternating signal may be applied to the second electrode unit. The first electrode unit may be configured so the first electrode unit may be electrically connected to the object. An electrical alternating signal may be applied to the first electrode unit.

The first and the second electrode units together form a capacitive sensor unit for determining a distance between the first electrode unit and the second electrode unit. An electrical alternating signal may be applied to either the first or the second electrode unit.

The determining unit for determining the distance based on a current measured at the first electrode unit or the second electrode unit is connected to the first electrode unit and/or the second electrode unit. The output unit for outputting an output value based on the measured current may be connected to the determining unit.

The present embodiments also relate to a method for measuring a distance between a functional unit and an object (e.g., an examination object) with a medical engineering apparatus. The method includes providing a first electrode unit and a second electrode unit arranged separately therefrom. The first electrode unit and the second electrode unit together form a capacitive sensor unit for determining a distance between the object and the second electrode unit. The first electrode unit is configured so the first electrode unit may be connected to the object, and the second electrode unit is configured in connection with a functional unit that may move relative to an object. The method includes determining the distance based on a current measured at the first electrode unit or the second electrode unit, and outputting an output value based on the measured current.

In the act of providing, a first (e.g., planar) electrode unit and a second (e.g., planar) electrode unit arranged separately therefrom is provided. Together, the first and the second electrode units form a capacitive sensor unit for determining a distance between the object or the first electrode unit and the second electrode unit. The first electrode unit is configured so the first electrode unit may be connected to the examination object. The second electrode unit is configured in connection with a functional unit that may move relative to an examination object. The first electrode unit and the second electrode unit may be configured as a transmitter or receiver unit, so a transmitter unit-receiver unit pair form from the first electrode unit and the second electrode unit together. A current is measured at the receiver unit. The functional unit may be configured, for example, as an imaging unit or as a radiation unit.

In the act of determining, the distance is determined based on a current measured at the first electrode unit or the second electrode unit. In the act of outputting, an output value is output based on the measured current. The output value may be, for example, a warning, a removal, or a control signal to stop the functional unit. In one embodiment, the distance between the object and the functional unit may be determined contactlessly. In one embodiment, a collision of the functional unit with the object may be more reliably avoided.

The present embodiments also relate to a computer program product with a computer program that may be loaded directly into a memory facility of a control facility of a medical engineering apparatus, with program segments in order to carry out all acts of a method of the present embodiments when the computer program is executed in the control facility of the medical engineering apparatus.

The present embodiments also relate to a computer-readable medium (e.g., a non-transitory computer-readable storage medium) on which program segments that may be read-in and executed by a computer unit are stored in order to carry out all acts of the method of the present embodiments when the program segments are executed by the computer unit. The computer unit may be incorporated by the medical engineering apparatus.

### BRIEF DESCRIPTION OF THE DRAWINGS

FIG. 1 schematically shows a representation of an embodiment of a medical engineering apparatus;

FIG. 2 schematically shows a representation of an embodiment of a sensor unit in a first embodiment;

FIG. 3 schematically shows a representation of an embodiment of a sensor unit in a second embodiment;

FIG. 4 schematically shows a representation of an embodiment of an ECG recording system; and

FIG. 5 schematically shows a representation of an embodiment of a method.

### DETAILED DESCRIPTION

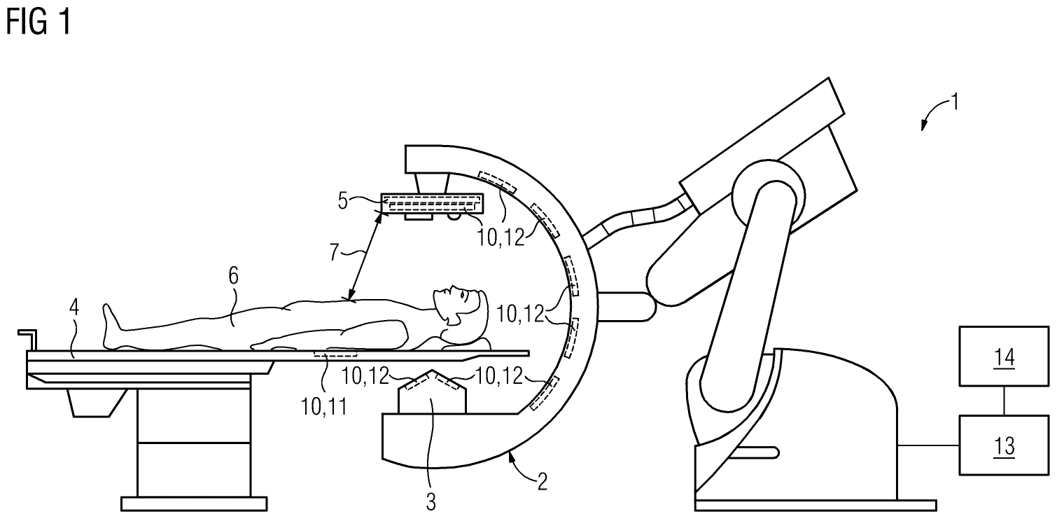

FIG. 1 shows an example embodiment of a medical engineering apparatus 1. The medical engineering apparatus 1 includes a functional unit 2 that may move relative to an object 6 (e.g., an examination object). The medical engineering apparatus 1 also includes a first electrode unit 11 and a second electrode unit 12 arranged separately from the first electrode unit 11. The first electrode unit 11 and the second electrode unit 12 together form a capacitive sensor unit 10 for determining a distance 7 between the object 6 or the first electrode unit 11 and the second electrode unit 12, with the first electrode unit 11 being configured to be electrically connected to the object 6 and the second electrode unit 12 being configured in connection with the functional unit 2. An electrical alternating signal may be applied to the first electrode unit 11 or the second electrode unit 12. The medical engineering apparatus 1 also includes a determining unit 13 for determining the distance 7 based on a current measured at the first electrode unit 11 or the second electrode unit 12, with the determining unit 13 being connected to the first electrode unit 11 or/and the second electrode unit 12. The medical engineering apparatus 1 also includes an output unit 14, connected to the determining unit 13, for outputting an output value based on the measured current.

In a first embodiment, the first electrode unit 11 is configured as a transmitter unit, and the second electrode unit 12 is configured as a receiver unit. In a second embodiment, the first electrode unit 11 is configured as a receiver unit, and the second electrode unit 12 is configured as a transmitter unit. In a combined embodiment, the receiver unit may be changed into a transmitter unit, and the transmitter unit may be changed into a receiver unit.

In one embodiment, the functional unit 2 is an imaging unit (e.g., a C-arm with an X-ray source 3 and an X-ray detector 5) or a radiation unit.

The determining unit 13 determines an overcoupling strength and/or a current discharging to ground. The determining unit 13 may determine a current using a current measuring unit, a distance using a distance determining unit, and/or a transmitter unit using an identification unit.

A plurality of second electrode units 12 are configured in connection with the functional unit 2. Each second electrode unit 12 of the plurality of second electrode units 12 is configured as a conductive element. A signal (e.g., a voltage) is applied or may be applied to the first electrode unit 11 or the at least one second electrode unit 12. Signals of different frequency or with different impressed codes are applied or may be applied to first or second electrode units 11, 12 configured as separate transmitter units. The signal has a frequency of less than 1 MHz or less than 100 Hz.

The first electrode unit 11 is arranged as a pad under the object 6 or on the object 6, integrated as a pad in a patient couch 4, or configured as a flexible pad. In FIG. 1, the pad is integrated in the patient couch by way of example. The first electrode unit 11 may be incorporated by an ECG recording system.

In an embodiment, the medical engineering apparatus 1 also has a conductive layer, to which a second current may be applied, on the functional unit 2 for collision detection.

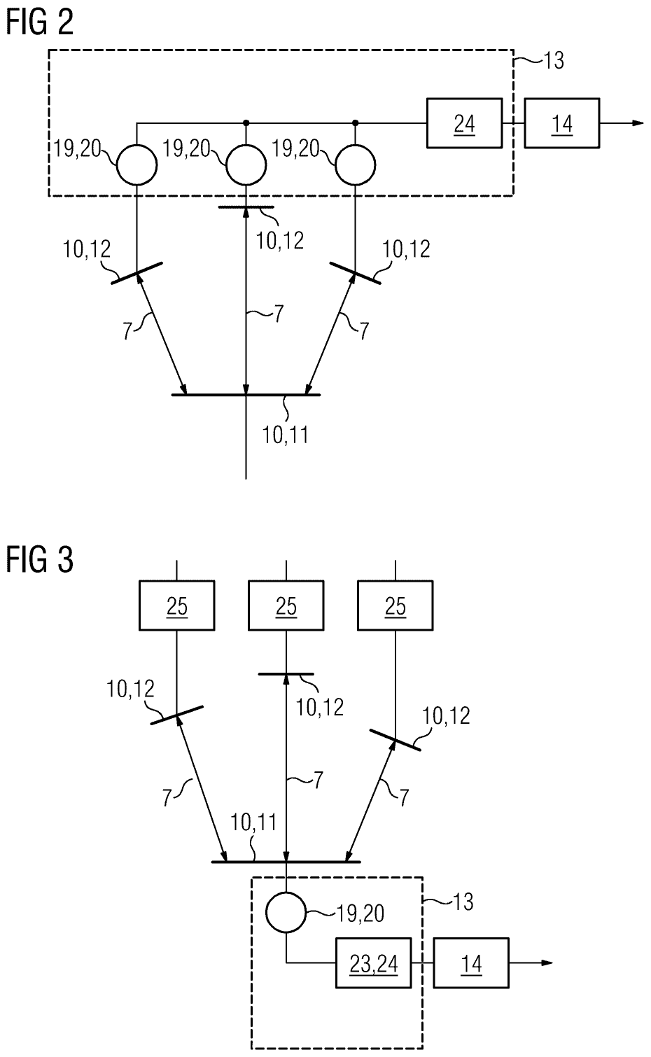

FIG. 2 shows an example embodiment of a sensor unit 10 in a first embodiment. In the first embodiment, the first electrode unit 11 is configured as a transmitter unit, and the second electrode unit 12 is configured as a receiver unit. The receiver unit or the second electrode units 12 are connected to the determining unit 13.

The determining unit 13 may determine a current using a current measuring unit 19, 20 and/or may determine a distance 7 using a distance determining unit 24 between a transmitter unit (e.g., the first electrode unit 11 or the object connected to the first electrode unit 11) and a receiver unit (e.g., a second electrode unit 12). The distance 7 may be determined for every other electrode unit 12, which are each a receiver unit. The distance 7 may be a distance between the first electrode unit 11 (e.g., the object surface) and the second electrode unit 12. The object or its surface may act as a transmitter unit due to the conductive connection of the first electrode unit 11 to the object.

FIG. 3 shows an example embodiment of a sensor unit 10 in a second embodiment. In the second embodiment, the first electrode unit 11 is configured as a receiver unit, and the second electrode units 12 are configured as transmitter units. The receiver unit or the first electrode unit 11 is connected to the determining unit 13.

The determining unit 13 may determine a current using a current measuring unit 19, 20, determine a distance 7 using a distance determining unit 24 between a transmitter unit (e.g., the second electrode units 12) and a receiver unit (e.g., the first electrode unit 11 or the object connected to the first electrode unit 11), and/or determine a transmitter unit using an identification unit 23.

Every other electrode unit 12 may be connected to an impressing unit 25, where signals of different frequency or with different impressed codes are applied or may be applied to different second electrode units 12.

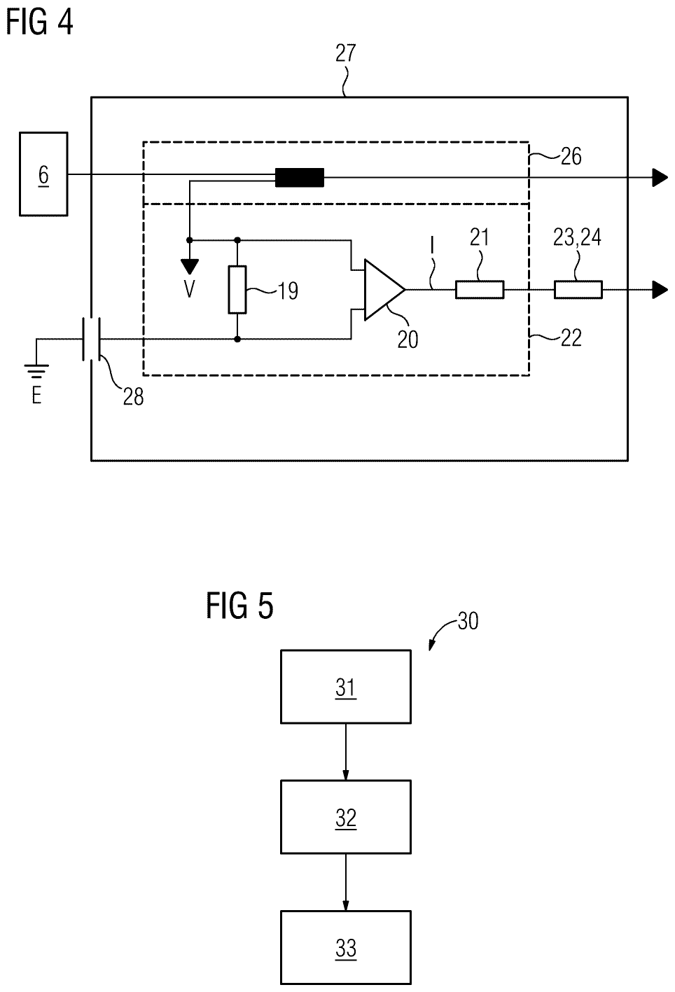

FIG. 4 shows an example embodiment of an ECG recording system 27. The ECG recording system 27 includes an ECG measuring unit 26 that is connected to the object 6. The ECG recording system 27 also includes an interference signal path 22 with a current measuring unit 19, 20. The current flowing from an internal reference potential V of the ECG device 27 via a capacitive coupling to an external fixed reference potential E, the ground potential E, is measured with this current measuring unit 19, 20. This measured interference signal I is primarily common mode interference signals. To make a defined interference signal path 22 available for this interference signal I, at which the interference signal I may be easily measured, a more extensive conductive surface 28 (e.g., in the form of a metal plate or a foil) is connected to the internal reference potential V of the ECG device 27, which forms a “capacitor surface” or a capacitor to the ground potential E. The current measuring unit 19, 20 is wired between the internal reference potential V and the ground potential E in this interference signal path 22.

For the current measuring unit 19, 20, a current sensing resistor 19 (e.g., a shunt resistor) wired between internal reference potential V and the ground potential E, and a voltage measuring facility 20 wired parallel to the shunt resistor 19 are used for measuring the current on the interference signal path 22. The second voltage measuring facility 20 may again be implemented by an amplifier (e.g., by a PGA). The measured interference signal or the measured current I is detected by an interference signal detecting unit 21 wired to the output of the voltage measuring facility 20, is digitized, for example by an A/D converter, and processed further in the determining unit 13. The distance 7 may be determined by the determining unit 13.

FIG. 5 shows an example embodiment of a method 30. The method 30 for measuring the distance between a functional unit and an object (e.g., an examination object) with an embodiment of a medical engineering apparatus has the acts of providing 31, determining 32, and outputting 33 (e.g., in this order). In the act of providing 31, a first electrode unit and a second electrode unit arranged separately from the first electrode unit are provided. The first electrode unit and the second electrode unit together form a capacitive sensor unit for determining a distance between the object or the first electrode unit and the second electrode unit. The first electrode unit is configured to be connected to the examination object, and the second electrode unit is configured in connection with a functional unit that may move relative to an examination object. In the act of determining 32, the distance is determined based on a current measured at the first electrode unit or the second electrode unit. In the act of outputting 33, an output value is output based on the measured current.

Although the invention has been illustrated in detail by the example embodiments, the invention is not limited by the disclosed examples, and a person skilled in the art can derive other variations herefrom without departing from the scope of the invention.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical engineering apparatus comprising:
a functional unit that is movable relative to an object;
a first electrode unit and a second electrode unit arranged separately from the first electrode unit, the first electrode unit and the second electrode unit together forming a capacitive sensor unit configured to determine a distance between the object and the second electrode unit, wherein the first electrode unit is configured to be electrically connectable to the object, and the second electrode unit is configured in connection with the functional unit, and wherein an electrical alternating signal is applicable to the first electrode unit or the second electrode unit;
a determining unit configured to determine the distance based on a current measured at the first electrode unit or the second electrode unit, wherein the determining unit is connected to the first electrode unit, the second electrode unit, or the first electrode unit and the second electrode unit; and
an output unit configured to output an output value based on the measured current.

2. The medical engineering apparatus of claim 1, wherein the object is an examination object.

3. The medical engineering apparatus of claim 1, wherein the first electrode unit is configured as a transmitter unit, and the second electrode unit is configured as a receiver unit.

4. The medical engineering apparatus of claim 1, wherein the first electrode unit is configured as a receiver unit, and the second electrode unit is configured as a transmitter unit.

5. The medical engineering apparatus of claim 3, wherein the receiver unit is changeable into a transmitter unit, and the transmitter unit is changeable into a receiver unit.

6. The medical engineering apparatus of claim 1, wherein the functional unit is an imaging unit or a radiation unit.

7. The medical engineering apparatus of claim 6, wherein the functional unit is the imaging unit, the imaging unit comprising a C-arm with an X-ray source and an X-ray detector.

8. The medical engineering apparatus of claim 1, wherein the determining unit is further configured to determine an overcoupling strength, a current discharging to ground, or the overcoupling strength and the current discharging to ground.

9. The medical engineering apparatus of claim 1, further comprising a plurality of second electrode units configured in connection with the functional unit,
wherein each second electrode unit of the plurality of second electrode units is configured as a conductive element.

10. The medical engineering apparatus of claim 9, wherein a signal is applied to the first electrode unit or at least one second electrode unit of the second electrode unit and the plurality of second electrode units.

11. The medical engineering apparatus of claim 10, wherein the signal is a voltage.

12. The medical engineering apparatus of claim 10, wherein signals of different frequency or with different impressed codes are applied to electrode units of the first electrode unit, the second electrode unit, and the plurality of second electrode units, the electrode units being configured as separate transmitter units.

13. The medical engineering apparatus of claim 10, wherein the signal has a frequency less than 1 MHz.

14. The medical engineering apparatus of claim 13, wherein the signal has a frequency less than 100 Hz.

15. The medical engineering apparatus of claim 1, wherein the first electrode unit is arranged as a pad under the object or on the object, is integrated as a pad in a patient couch, or is configured as a flexible pad.

16. The medical engineering apparatus of claim 1, wherein the first electrode unit is incorporated by an electrocardiogram (ECG) recording system.

17. The medical engineering apparatus of claim 1, further comprising a conductive layer, to which a second current applicable, the conductor layer being on the functional unit for collision detection.

18. A method for measuring a distance between a functional unit and an object with a medical engineering apparatus, the method comprising:
providing a first electrode unit and a second electrode unit arranged separately from the first electrode unit, the first electrode unit and the second electrode unit together forming a capacitive sensor unit configured to determine a distance between the object and the second electrode unit, wherein the first electrode unit is configured to be connectable to the object, and the second electrode unit is configured in connection with the functional unit, which is movable relative to the object, wherein an electrical alternating signal is applicable to the first electrode unit or the second electrode unit;
determining the distance based on a current measured at the first electrode unit or the second electrode unit; and
outputting an output value based on the measured current.

19. In a non-transitory computer-readable storage medium that stores instructions executable by one or more processors of a medical engineering apparatus to measure a distance between a functional unit and an object, the instructions comprising:
providing a first electrode unit and a second electrode unit arranged separately from the first electrode unit, the first electrode unit and the second electrode unit together forming a capacitive sensor unit configured to determine a distance between the object and the second electrode unit, wherein the first electrode unit is configured to be connectable to the object, and the second electrode unit is configured in connection with the functional unit, which is movable relative to the object, wherein an electrical alternating signal is applicable to the first electrode unit or the second electrode unit;
determining the distance based on a current measured at the first electrode unit or the second electrode unit; and
outputting an output value based on the measured current.
